(19) United States
(12) Patent Application Publication
(10) Pub. No.: US 2010/0047733 A1
(43) Pub. Date: Feb. 25, 2010

(54) Device, system and method for procedures associated with the intra-oral cavity

(75) Inventor: Oded Nahlieli, Ashkelon (IL)

Correspondence Address: THE NATH LAW GROUP, 112 South West Street, Alexandria, VA, 22314, US

(73) Assignee: SIALO-LITE LTD., Ashkelon (IL)

(21) Appl. No.: 12/461,626

(22) Filed: Aug. 18, 2009

Related U.S. Application Data
(63) Continuation-in-part of application No. 11/792,040, filed on Jun. 1, 2007.

(30) Foreign Application Priority Data
Jul. 12, 2005 (IL) 169641

(51) Int. Cl. A61B 1/24; A61B 1/018; A61B 1/04; A61C 19/04

(52) U.S. Cl. 433/29; 600/104; 600/109; 433/215

(57) ABSTRACT

A device and system for use in procedures associated with the intra-oral cavity, the device having a probe member and a handpiece for holding the elongate probe member, the probe member having an elongate distal portion capable of being manipulated in the intra-oral cavity, the probe member comprising: at least one treatment channel having a distal opening in the elongate distal portion, the at least one treatment channel being configured for enabling operation of a suitable tool via the distal opening; at least one illumination channel comprising a first light guide having a first proximal end configured for optical coupling to a light source system, and a second distal end in the distal portion for illuminating regions of interest during operation of the device; at least one light collection channel configured for optical coupling to an imaging system and for collecting and transmitting light reflected from the regions of interest during operation of the device.

This is a Continuation-In-Part Application of U.S. patent application Ser. No. 11/792,040, filed Jun. 1, 2007, under 35 U.S.C. 371 as a national stage of PCT/IL2006/00804, filed Jul. 12, 2006, claiming the benefit of Israeli Application No. 169641, filed Jul. 12, 2005, the entire content of each of which is hereby incorporated by reference in its entirety.

### FIELD OF THE INVENTION

This invention relates to devices, systems and methods for procedures associated with the intra-oral cavity and dentistry in general, and in particular in the treatment of root canals, in dental implant procedures, and in sinus augmentation procedures.

### BACKGROUND OF THE INVENTION

Conventional root canal treatment techniques are based on drilling through the top of the tooth in the case of molars, or via the lingual side or palatinal side of the tooth in the case of anterior teeth, and removing the infected pulp. The root canal is then subjected to several therapeutic procedures including extirpation of the root pulp, cleaning and clearing the canal, widening the canal and smoothing its walls by removing hard tissue obstacles, treating it to remove infection pathogens, washing, drying and filling. One or more x-rays of the root are required to provide feedback on the process. Dental drills and endodontic files are commonly employed for at least some of these procedures.

Conventional dental implant procedures for the upper jaw or maxilla sometimes requires a sinus augmentation procedure to be first implemented, so as to provide sufficient bone to anchor the implant. A commonly used sinus augmentation procedure is performed from inside the inside the intraoral cavity. In what is known as the lateral approach, a lateral incision is made into the gum and gum tissue is pulled back, and an opening is cut in the exposed lateral boney wall of the sinus. The sinus is covered by a thin membrane, which is lifted away to create a space into which allogenic, autogenous, or synthetic bone graft material is inserted via the opening. However, cutting through the boney wall and lifting the membrane can lead to tearing or puncturing of the membrane if not done carefully, and this requires the membrane to be immediately repaired, postponing the sinus augmentation procedure until healing of the membrane is complete. Once the bone graft material has been is integrated in the maxilla, which can normally take between 6 and 12 months, the dental implant can be installed, followed by the dental prosthesis.

Another method sometime used when there is a minimum of 5 mm involves inserting the bone graft material from the crest of the bone during implant placement, and is known as the crestal approach. The normal implant hole is drilled until about 1 mm before the sinus, and then another tool is used for taping the remaining shell of bone towards the sinus. The tapped shell displaces the membrane into the sinus, making room for the graft material which is then inserted via the implant hole. The implant is then installed into the prepared bone, allowing the graft material to become integrated while anchoring the implant in situ.

By way of general background, lasers are known for use in general surgery and dentistry, and are sometimes employed for the material removing process.

For example, U.S. Pat. No. 6,162,052 discloses a laser handpiece configured by a main body and a laser probe mounted on the tip end of the main body. The laser probe has an emission fiber, and a laser beam generated by a laser beam source is emitted from an emission end portion of the emission fiber. The emission end portion of the emission fiber is formed into a circular conical shape. The laser beam emitted from the emission end portion contains a first laser beam which is emitted in the axial direction of the emission fiber, and a second laser beam which is emitted in a ring-like shape in a radial direction of the emission fiber.

U.S. Pat. No. 6,458,120 discloses a surgical laser system combining a laser diode array remotely connected to a hand-held surgical probe by a fiber bundle. The surgical probe includes a laser head which produces a laser beam for surgical tissue ablation that is delivered through a disposable intraocular probe tip. The probe tip is made of a short section of optical fiber. Auxiliary water and thermal electric cooling integral to the hand piece cools the laser head.

U.S. Pat. No. 6,679,837 discloses a medical surgical or dental laser light irradiating apparatus which enables operators as well as third persons to view the treatment. The medical laser light irradiating apparatus includes an optical fiber for emitting from its front end the laser light, a handpiece for holding said optical fiber, a relatively large camera and light incident face which is made integrally with the handpiece so that it faces at least a laser light irradiated area and is spaced therefrom, imaging means for imaging of at least laser light irradiated area based upon the light which is received through the light incident face, display means for displaying a result of imaging from said imaging means, and jetting means for jetting an air to an area in front of said light incident face. By way of general background, other imaging systems are known, for example in U.S. Pat. No. 6,013,025, which discloses a miniaturized endoscope formed by passing light in both directions down the endoscope.

Further by way of general background, WO 2009/024107 discloses a modular endoscope system in which a plurality of connecting pieces, tools and cannulas are assigned to a single lens and are each selected according to the use thereof. The connecting pieces and the cannulas are said to be designed preferably for single use so that only the lens must be decontaminated.

### SUMMARY OF THE INVENTION

According to one aspect of the invention, there is provided a method for conducting a procedure in the intra-oral cavity comprising implementing the procedure using at least one suitable tool having a working end, and monitoring the procedure in real time using a suitable imaging system comprising an image acquisition unit, wherein said tool working end and said image acquisition unit are included in a handheld device, and wherein said image acquisition unit and said tool working end are positioned in the hand held device such said tool working end is in the field of view of said image acquisition unit at least during operation of the tool.

The procedure may be any suitable medical, dental or surgical procedure, and may comprise a tissue removal operation.

In at least some embodiments, the step of implementing the procedure may includes using, in turn, each one of a plurality of said tools, each said tool having a respective working end. Thus, at least one tool has its respective working end in the field of view of the image acquisition unit at least during operation of the respective tool.

Thus, the zone being operated or treated can be monitored in real time by the user of the device, providing effective control on surgery/treatment. Optionally, the zone may also magnified by the imaging system, allowing even more control.

In one application of the method, the said procedure is a sinus augmentation procedure applied to a maxilla comprising gum tissue and having a sinus lined with a sinus membrane, the method comprising the steps of:

(a) forming a window in the gum tissue in the maxilla;

(b) providing said device with a first said tool having a first tool working end that is configured for removing tissue;

(c) using said first tool end, removing material from the maxilla to form a channel extending from said window to said sinus membrane, while monitoring said material removal via said image acquisition unit;

(d) providing via said channel with suitable bone graft material in a space between the sinus and the sinus membrane to provide the required sinus augmentation.

In at least one embodiment of the method, prior to step (d), said space is formed by displacing said sinus membrane away from said channel. For example, said sinus membrane may be displaced away from said channel by inflating a suitable balloon at or near a distal end of said channel. The said balloon may constitute said working end of a second said tool, and said balloon may be transparent or translucent. The balloon inflation procedure may be monitored in real time via said image acquisition unit. Subsequent to said displacement of said membrane and prior to step (d), said balloon may be deflated and removed.

Step (d) may be implemented in a number of different ways. For example, step (d) may comprise providing a sponge in said space impregnated with said bone graft material. The sponge may be manipulated into position in said space via a third. said tool having a respective third said working end comprising a grasping function, wherein said sponge is releasably grasped by said third working end until seated in said space. For example, the sponge comprises a collagen matrix, and is pre-impregnated or impregnated in situ in said space.

Alternatively, step (d) may comprise injecting said bone graft material in said space. A fourth said tool may be provided in the form of a syringe having a respective fourth said working end in the form of a needle opening, and wherein said fourth tool is used for injecting said bone graft material into said space via said needle opening, and wherein said injection process is monitored in real time via said image acquisition unit.

Furthermore, step (d) may further includes filing said channel with suitable bone graft material, and further comprises the step of closing said window and allowing the bone graft material to integrate with the maxilla and heal. This may be followed with the step of installing a suitable dental implant in said maxilla such that the dental implant is anchored in said sinus augmentation, and thereafter a suitable prosthesis may be mounted to the implant.

Alternatively, step (d) may further include partially filing said channel with suitable bone graft material, and concurrently installing a suitable dental implant in said sinus augmentation, and further comprises the step of closing said window around the implant, and allowing the bone graft material to integrate with the maxilla and heal while concurrently anchoring the dental implant in said sinus augmentation. Thereafter, a suitable prosthesis may be mounted to the implant.

Clearly, the sinus augmentation procedure may be sufficient for one implant or for a series of adjacent implants, mutatis mutandis. In the latter case, it is possible for the same channel to be used as access for providing sinus augmentation for a row of missing teeth. Furthermore, a plurality of separate sinus augmentation procedures may be provided for the same patient.

In step (a), said window may be formed on a crest portion of the gum, and allows a relatively narrow channel to be formed to gain access to the sinus membrane.

In another application of the method, the said procedure is a dental implant procedure for a mandibular jaw, and comprises the steps of:
providing said device with a first said tool having a first tool working end that is configured for removing tissue;
using said first tool end, removing material from the mandibular jaw to form a bore configured for receiving the dental implant, while monitoring said material removal via said image acquisition unit.

This application of the method may further comprise checking the bore as it is being formed for evidence of possible perforation of the respective mandibular canal, and halting said material removal process to minimize or avoid damaging the respective inferior alveolar nerve.

In another application of the method, the said procedure is a root canal procedure, and comprises the steps of:
providing said device with a first said tool having a first tool working end that is configured for removing tissue;
using said first tool end, removing pulp material from the respective root canal, while monitoring said pulp material removal via said image acquisition unit.

According to another aspect of the invention, there is provided a device for use in procedures associated with the intra-oral cavity, the device comprising:
a probe member and a handpiece for holding said elongate probe member, said probe member having an elongate distal portion capable of being manipulated in the intra-oral cavity, said probe member comprising:
at least one treatment channel having a distal opening in said elongate distal portion, said at least one treatment channel being configured for enabling operation of a suitable tool via said distal opening, such that in operation of the device, a working portion of said tool projects distally from a distal end of said distal portion,;
at least one illumination channel different from said at least one treatment channel and comprising a first light guide having a first proximal end configured for optical coupling to a light source system, and a second distal end in said distal portion for illuminating regions of interest associated with the intra-oral cavity during operation of said device;
image acquisition unit configured for optical coupling to an imaging system, and further configured for collecting and transmitting light reflected from said regions to said proximal end during operation of said device;
wherein said tool working portion is in the field of view of said image acquisition unit at least during operation of the device.

In at least some embodiments, the handpiece comprises a longitudinal axis set at an angle α with respect to a longitudinal axis of said probing member, wherein said angle α is between about 45° and about 135°.

In at least some embodiments, the said field of view is greater than about 90 degrees. For example, said field of view may be about 120 degrees or about 180 degrees.

In at least some embodiments, said imaging acquisition unit may comprise a CCD or the like located in said probe member at or near said distal end.

In at least some embodiments, the device may comprise a said tool accommodated in said treatment channel wherein said working portion of said tool projects distally from a distal end of said distal portion.

In at least some embodiments of the invention, the distal end of said probe member may be configured as a tool, in particular a cutting or material removal tool, and during operation of said distal end as a tool, the image acquisition unit is positioned in the device such that said distal end is in the field of view of said image acquisition unit. For example, the distal end may comprise a sharp, serrated or abrasive edge that may allow the distal end to cut through and/or remove tissue material as the device is manipulated and moved against the tissue. This edge may project distally beyond the distal end or the image acquisition unit may comprise a 180 degree wide angle field of view, or the image acquisition unit may be retracted proximally in the distal end, such that the edge is within the field of view of the image acquisition unit. At least the distal end of the probe member may optionally be made from a transparent material. The probe member may be made from a disposable material for disposal of the probe member after use with a single patient.

According to another aspect of the invention there is provided a system for use in procedures associated with the intra-oral cavity, comprising:

(A) a device comprising a probe member and a handpiece for holding said elongate probe member, said probe member having an elongate distal portion capable of being manipulated in the intra-oral cavity, said probe member comprising:
at least one treatment channel having a distal opening in said elongate distal portion, said at least one treatment channel being configured for enabling operation of a suitable tool via said distal opening, such that in operation of the device, a working portion of said tool projects distally from a distal end of said distal portion,;
at least one illumination channel different from said at least one treatment channel and comprising a first light guide having a first proximal end configured for optical coupling to a light source system, and a second distal end in said distal portion for illuminating regions of interest associated with the intra-oral cavity during operation of said device;
image acquisition unit configured for collecting and transmitting light reflected from said regions to said proximal end during operation of said device;
wherein said tool working portion is in the field of view of said image acquisition unit at least during operation of the device.

(B) a light source system coupled to said proximal end of said first illumination system;

(C) an imaging system comprising a display unit coupled to said image acquisition unit;

(D) a said tool accommodated in said treatment channel wherein said working portion of said tool projects distally from a distal end of said distal portion.

In at least some embodiments, the handpiece comprises a longitudinal axis set at an angle α with respect to a longitudinal axis of said probing member, wherein said angle α is between about 45° and about 135°.

In at least some embodiments, the said field of view is greater than about 90 degrees. For example, said field of view may be about 120 degrees or about 180 degrees.

In at least some embodiments, said imaging acquisition unit may comprise a CCD or the like located in said probe member at or near said distal end.

According to at least another aspect of the invention, a device is provided for use in a root canal treatment, the device comprising:

a probe member and a handpiece for holding said elongate probe member, said probe member having an elongate distal portion capable of being accommodated in a root canal, said probe member comprising:

at least one treatment channel having a distal opening in said elongate distal portion, said at least one treatment channel being configured for enabling operation of a suitable root canal treatment tool via said distal opening;

at least one illumination channel comprising a first light guide having a first proximal end configured for optical coupling to a light source system, and a second distal end in said distal portion for illuminating internal regions of the root canal during operation of said device;

at least one light collection channel comprising a second light guide having a first proximal end configured for optical coupling to an imaging system, and a second distal end located in said distal portion for collecting and transmitting light reflected from internal regions of the root canal to said proximal end during operation of said device.

The said elongate distal portion may comprise a length dimension along an elongate direction generally correlated with a depth of a root canal, and the length dimension may be, for example, between about 10 mm and about 30 mm. The elongate distal portion may comprises a distal tip having a transverse dimension in a direction orthogonal to said elongate direction generally smaller than a width of a root canal at the apex thereof. The elongate distal portion may comprise a said transverse dimension that is generally uniform along said elongate dimension thereof. For example, the transverse dimension may be between about 0.5 mm and about 1.5 mm. Alternatively, the elongate distal portion comprises a said transverse dimension that varies along said elongate dimension thereof. For example, the transverse dimension may vary between about 0.5 mm and about 1.5 mm along said elongate dimension thereof.

Optionally, the distal opening, the distal end of said first light guide, and the distal end of said second light guide are at or proximate to said distal tip. Optionally, the elongate distal portion has an enlarged portion proximately spaced from said tip by a first spacing, and the first spacing may comprise a length dimension along an elongate direction generally correlated with between about 50% and about 70% of a depth of a root canal including the tip thereof. In such an embodiment, the distal opening and said distal end of said first light guide may be at or proximate to said distal tip, and the distal end of said second light guide may be comprised in said enlarged portion. Alternatively, the distal opening may be at or proximate to said distal tip, and the distal end of said first light guide and said distal end of said second light guide are comprised in said enlarged portion.

The treatment channel may comprise a suitable bore adapted for accommodating and enabling operation of any one of: a laser tool, a dental drill, a dental file, an ultrasonic tool, an RHF cutting tool, a reaming tool, a grasping tool, a syringe needle or the like. Optionally, the treatment channel is substantially rectilinear and comprises a proximal opening in said device substantially longitudinally opposed to said distal opening.

The device may optionally comprise the light source system optically coupled to said first light guide. The light system may optionally comprise at least one LED, for example.

Optionally, the first light guide comprises at least one of: at least one optical fiber; any suitable optical waveguide arrangement.

The device may optionally comprise the imaging system optically coupled to said second light guide. Optionally, the imaging system comprises a CCD or the like in optical communication with said distal end of said second light guide via a system of lenses. The CCD or the like may be operatively connectable to a microprocessor unit or the like adapted for processing images transmitted thereto by said CCD or the like. Optionally, the second light guide comprises an optical folding member.

Alternatively, the second light guide comprises at least one optical fiber.

Optionally, the at least one illumination channel is integral with said at least one light collection channel, said corresponding first waveguide being integral with said second light collection channel.

The device may optionally comprise at least one auxiliary channel having a first proximal end configured for operative connection to at least one of a fluid source and a vacuum source, and a second distal end in said distal portion for providing fluid or a vacuum, respectively, to internal regions of the root canal during operation of said device. For example, the fluid source may comprise any one of a liquid or air source.

Optionally, the handpiece comprises a longitudinal axis set an angle α with respect to a longitudinal axis of said probing member. By way of example, angle α may be between about 45° and about 135°.

According to another aspect of the invention, a system for root canal treatment is provided, comprising

a device according to the first aspect of the invention, comprising any combination of features therein;

a light source system coupled to said proximal end of said first illumination system;

an imaging system coupled to said proximal end of said second illumination system;

a root canal treatment tool comprised in said treatment channel, said tool having a treating portion associated with said distal outlet.

The tool may comprise, for example, any one of: a laser tool, a dental drill, a dental file, an ultrasonic tool, an RHF cutting tool, a reaming tool, a grasping tool, a syringe needle or the like. Optionally, the tool is a laser tool having a laser transmission end adapted for any one of axial transmission of a laser beam and non-axial transmission of a laser beam, with respect to a longitudinal axis of said elongate distal portion. Alternatively, the tool is a syringe needle coupled to at least one of a source of filling agent and a source of sealing agent for respectively filling or sealing a root canal during operation of the device.

According to another aspect of the invention a method is provided for monitoring a root canal treatment, comprising:

illuminating the root canal with an illumination radiation and acquiring at least one image of said root canal illuminated thereby, in association with a root canal treatment.

Optionally, the illumination and image acquisition are carried out by means of a suitable probe member inserted into said root canal. This may optionally be carried out using the system according to the second aspect of the invention.

Optionally, the image acquisition may be carried out while performing a root canal treatment.

Optionally a plurality of images may be acquired to provide a video stream in real time.

According to another aspect of the invention, a method is provided for treating a root canal, comprising

illuminating the root canal with an illumination radiation and acquiring at least one image of said root canal illuminated thereby; and

performing a root canal treatment.

The image acquisition may be carried out while performing a root canal treatment. Optionally, a plurality of images is acquired to provide a video stream in real time. Optionally, illumination and said image acquisition may be carried out by means of suitable probe member inserted into said root canal. Optionally, the method may be carried out using a system according to the second aspect of the invention. Optionally, the root canal treatment may comprise one of a sealing or filing procedure for the root canal. Optionally, said one of a sealing or filing procedure comprises injecting a suitable sealant or filler, respectively, to the root canal via said treatment channel.

### BRIEF DESCRIPTION OF THE DRAWINGS

In order to understand the invention and to see how it may be carried out in practice, some embodiments will now be described, by way of non-limiting example only, with reference to the accompanying drawings, in which:

FIG. 1 schematically illustrates the general anatomy of a tooth including a root canal.

FIG. 2 illustrates, in transverse cross-sectional view, a device according to a first embodiment of the invention.

FIG. 3 illustrates a longitudinal section of the embodiment of FIG. 2, taken along X-X.

FIG. 4 illustrates in partial transverse cross sectional view the embodiment of FIG. 2 comprising a tool.

FIG. 5 illustrates in partial transverse cross sectional view a variation of the embodiment of FIG. 2 comprising an integral tool.

FIG. 6 illustrates in transverse cross sectional view a variation of the embodiment of FIG. 2 comprising a non-rigid tool; FIG. 6a illustrates a variation in the tool of FIG. 6.

FIG. 7 illustrates in partial transverse cross sectional view another variation of the embodiment of FIG. 2 comprising a tool.

FIGS. 8a and 8b illustrate in partial transverse cross sectional view a number of variations of the probe member of the embodiment of FIG. 2.

FIG. 9 illustrates schematically a system according to one embodiment of the invention.

FIGS. 10a to 10c illustrate a use of a device according to an embodiment of the invention.

FIGS. 11a to 11e illustrate a procedure associated with the intra-oral cavity according to an embodiment of the invention.

FIG. 12 illustrates, in transverse cross-sectional view, a device according to a second embodiment of the invention.; FIG. 12a illustrates one mode of operation of the tip of the embodiment of FIG. 12

### DETAILED DESCRIPTION OF EMBODIMENTS

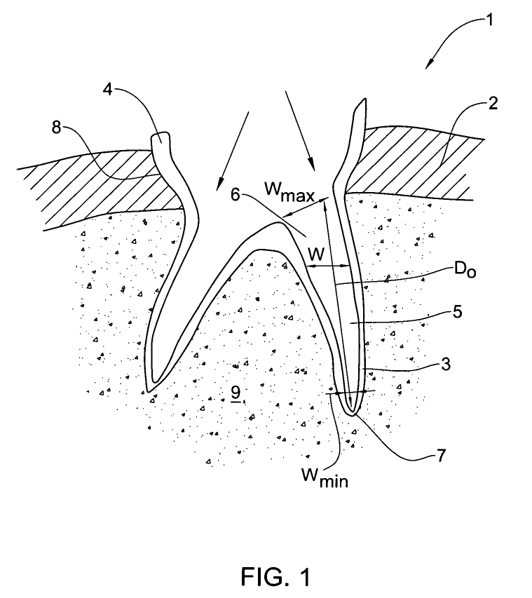

FIG. 1 illustrates the general anatomy of a tooth 1 such as a molar that is being prepared for a root canal treatment. Part of the crown 4 is removed by drilling or using any other suitable technique, for example, followed by removal of pulp so that only dentine 3 remains surrounding the root canal 5. The root 6 comprises a root apex 7 that projects into the jaw 9′. The root canal 5 comprises a depth D₀, which may be regarded as the linear length of the root 6 between the neck 8 of the tooth and the root apex 7, and width W which is typically the same or greater than the original diameter or width of the pulp space that is left when the pulp is removed therefrom. The width W typically diminishes in magnitude from a maximum diameter or width Wₘₐₓ the root opening 6 to a minimum diameter or width Wₘᵢₙ at the root apex 7, and thus the width W may be regarded as an average or median value, for example, of the root canal width or diameter for a given root canal.

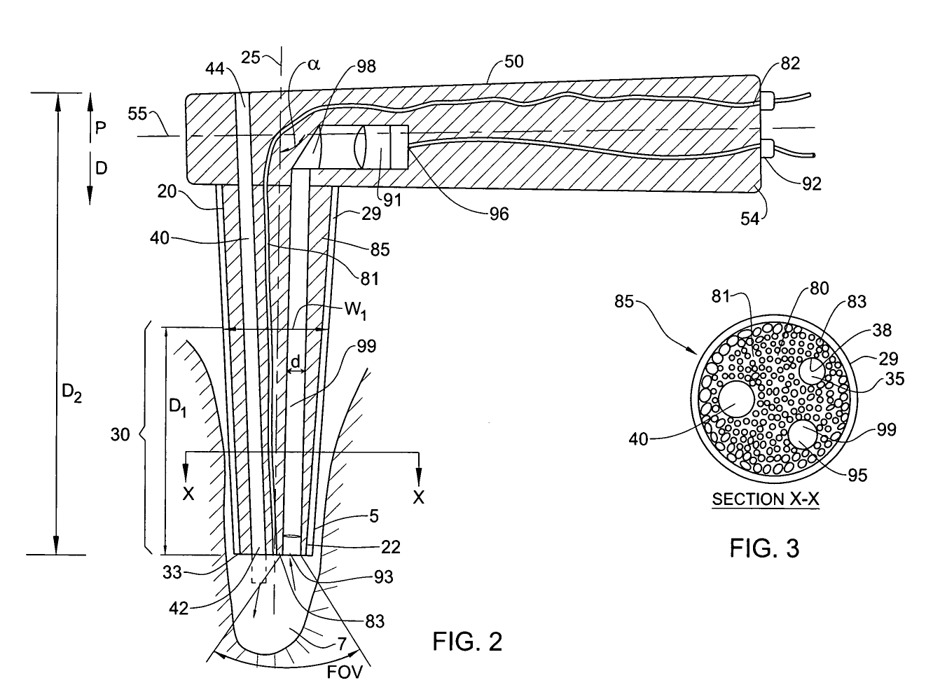

Referring to FIGS. 2 and 3, a device for use in procedure associated with the intra-oral cavity, according to a first embodiment of the invention, generally designated 100, comprises an elongate probe member 20, and a handle or handpiece 50 for holding the probe member 20. As will become clearer herein, while the device 100 according to this or other embodiments is initially described in the context of its use in a dental treatment, such as for example a root canal treatment, the device may also be used for other procedures associated with the intra-oral cavity, such as for example dental implant procedures, sinus augmentation treatments, and so on, in particular such procedures which comprise removing tissue or other material from the intra-oral cavity.

Herein, the intra-oral cavity refers interchangeably to the oral cavity, and is taken to include structures of the head which are accessible via the intra-oral cavity, for example root canals, sinuses, the jaws, etc.

Herein the term “distal” (D) relates to a direction generally away from the user of the device, while the term “proximal” (P) refers to a direction opposed to distal, that is, a direction generally towards the user of the device.

The probing member 20 comprises an elongate distal portion 30 that is capable of being manipulated in the intra-oral cavity, for example capable of being inserted and accommodated in at least a part of a root canal 5, in operation of the device 100. Accordingly, the elongate distal portion has an axial dimension D₁ in the distal direction that as a minimum is generally correlated to the depth into which it is desired to insert the probe member 20 with respect to the root canal, and may be as deep as the depth D₀, of the root canal 5 or greater. Dimension D₁ thus may be of similar or smaller dimension to D₀. Thus, for some applications of the device it is possible for the axial dimension D₁ to be smaller than the depth D₀, for example when the specific root canal tool being used (see below) has an effecting working depth that projects beyond the probe distal tip 22 towards the root apex 7.

While the depth D₀, may vary between individuals, and from tooth to tooth in any individual, between different roots of a single tooth, and further depend also on the age, sex and physical makeup of the individual among other factors, the depth D₀, may vary in most cases between about 15 mm and about 30 mm.

The shape and transverse dimension or width W₁ of the distal portion 30 may be uniform or may vary. For example, the distal portion 30 may be in the form of an elongate cylinder, the diameter or width W₁ being smaller than the corresponding width W of the root canal 5 at the depth to which is desired to introduce the probe member 20, to allow the distal portion 30 to be inserted therein with some clearance. Thus, if it is desired that the distal portion should be inserted to the root apex 7, then the diameter W₁ should be a little under the minimum width Wₘᵢₙ of the root canal 5. In such a case, W₁ may be, by way of example, between about 300 micron to about 500 micron or to about 700 micron, depending on the actual dimensions of the root canal with respect to which the device is being used. Alternatively, it may be sufficient for the distal portion to be introduced to between about 50% and about 70% or about 80% of the depth Do towards the apex 7, in which case W₁ may be, by way of example, between about 500 micron and about 700 micron over 700 micron, depending on the actual dimensions of the root canal with respect to which the device is being used. Alternatively, it may be sufficient for the distal portion to be introduced to between about 20% and about 35% or about 45% of the depth D₀ towards the apex 7, in which case W₁ may be, by way of example, between about 700 micron and about 1300 micron or about 1500 micron, depending on the actual dimensions of the root canal with respect to which the device is being used.

Alternatively, the distal portion may be in the form of an elongate frusto-cone, having a maximum diameter or width and a minimum diameter of width such as to enable the probe to be introduced to the desired depth into the root canal 5 while providing a lateral clearance therebetween. Thus, if it is desired that the distal portion 30 should be inserted to the root apex 7, then the minimum diameter at the distal end of the probe member 20 should be a little under the minimum width Wₘᵢₙ of the root canal 5, and the maximum diameter of the probe, at the proximal end of the distal portion 30, should also be less than the width Wₘₐₓ. The same applies, mutatis mutandis, when it is desired to limit the depth of insertion of the probe to less than up to the apex.

The mean or median width W typically varies between individuals, and from tooth to tooth in any individual, and within a tooth having multiple roots, and further depends also on the age, sex and physical makeup of the individual, among other factors.

The longitudinal dimension D₂ of the probe member 20, including the part of the handpiece 50 to which the probe member is connected, is such as to enable the device 100 to be inserted and manipulated in the oral cavity, and allow the distal portion 30 to be inserted towards and into the root canal 5.

For different applications of the device 100, different dimensions for D₁, D₂, W₁ may be required.

The probe member 20 may be rigid or semi rigid, having at least sufficient rigidity to enable the same to be manipulated into the root canal by handling the handpiece 50. The probe member 20 may thus be made from a suitable metal or hard plastic material, for example.

A longitudinal axis 25 may be defined for the probe member 20, taken along the elongate direction thereof. The hand piece 50 is configured for enabling the user to hold and manipulate the probe member 20, and may also comprise an elongate graspable member having its longitudinal axis 55 at an angle α to axis 25. Angle α may have any suitable angular value, by way of non-limiting example in the range of from about 45° and about 135°, including for example about 90° or about 125°, inter alia. In other embodiments, the angle may be about 0°, and thus the handpiece 50 may be in the form of as a wand.

In the illustrated embodiment, the angle α is fixed, though in other embodiments, this angle may be variable, the device 100 comprising a suitable pivoting or deflection mechanism (not shown) for rotating or bending the probe member 20 with respect to the handpiece 50, allowing the user greater flexibility in use of the device 100. The probe member 20 and the hand piece 50 may be integrally or otherwise joined to one another.

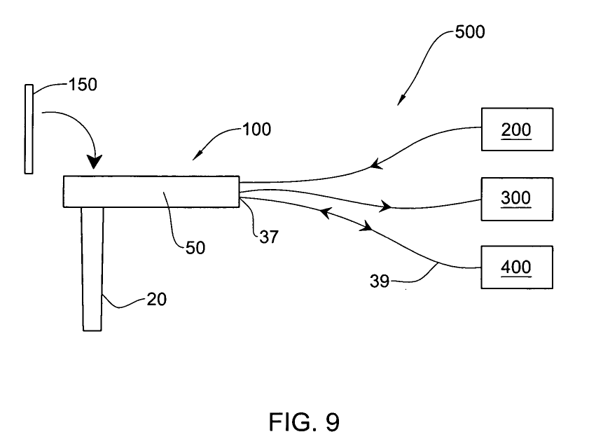

The device 100, and in particular the probe member 20, comprises at least one treatment channel 40 having a distal opening 42 at the tip of the distal portion 30, and the treatment channel 40 allows access to a tool, collectively designated as 150 (FIG. 9) to operate in the intra-oral cavity, for example on the root canal. In the illustrated embodiment the treatment channel is in the form of an elongate bore having a proximal opening 44 longitudinally opposed to the distal opening. By way of non-limiting example, the bore may comprise a diameter of between about 200 micron and about 500 micron. The proximal opening 44 allows any suitably shaped tool, in particular a rigid or semi rigid elongate tool, to be inserted into the treatment channel 40 such that a distal working end 91 of the tool may be projected into the root canal 5 via the distal opening 42. Such a tool, for example a dental file or reamer, which may be made from nickel titanium or stainless steel for example, may be operated manually from outside of the proximal opening 44.

Other tools, each of which may be used in turn via the working channel 40, may include, by way of non-limiting example, a laser energy delivery system, such as a laser cutting tool, a grasping tool such as micro tongs or a magnetized grasping tool, inter alia, wherein the working portion of the tool projects distally from the distal end of the device at least during operation of the device.

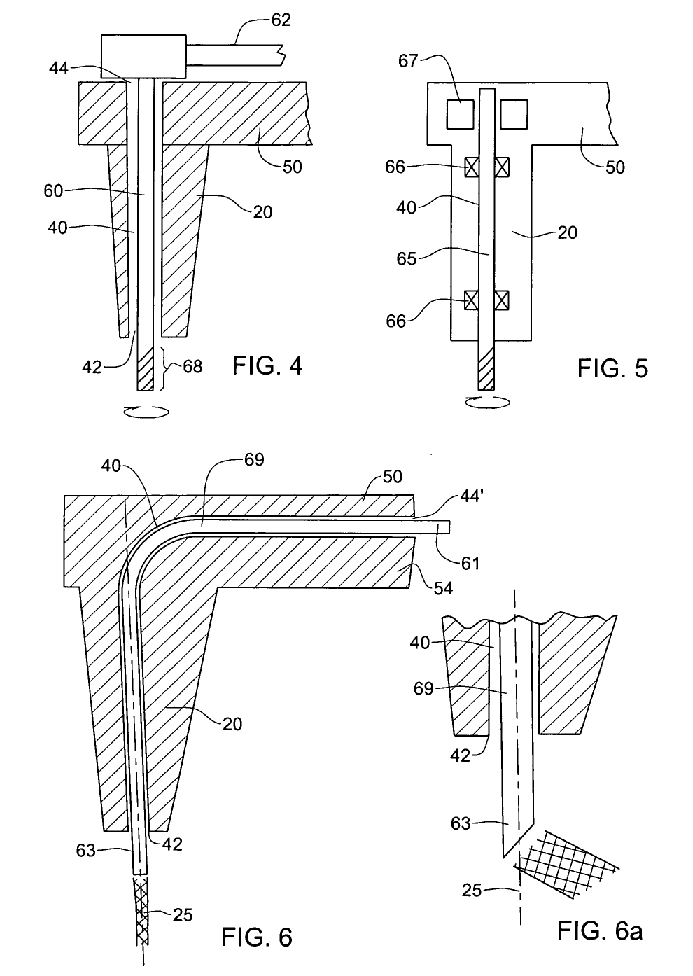

Alternatively, and referring to FIG. 4, such a tool 60 may be a powered tool, for example a dental drill or reamer, and the motor housing and handpiece 62 of the tool may be reversibly or permanently mounted onto the device 100 for use therewith. Such drills may comprise, for example, diamond drills or tungsten drills, and may be used for removing the crown, or for drilling into bone such as in the maxilla or mandible, for example. The working portion 68 of the tool, i.e. the portion of the tool that interacts with the root canal or other dental tissues, projects distally away from the distal end of the distal portion 30.

Alternatively, the tool may comprise a syringe having a syringe needle of suitable dimensions that may be inserted into the working channel 40 and optionally extend therethrough such that the working portion, i.e., the tip of the syringe needle comprising the syringe outlet, projects from the opening 42, and enables a desired agent to be delivered to the root via the syringe at least during operation of the device. Such an agent may comprise, by way of non-limiting example, irrigating solutions, antibiotics, liquid filler, liquid sealant, and so on, inter alia. The syringe needle may be substantially rigid, or may be flexible, depending on the general shape of the treatment channel 40.

Alternatively, and as illustrated in FIG. 5, a powered tool 65 may be integrated with the device 100, comprising suitable bearings 66 and a suitably powered micromotor 67.

In some variations of this embodiment, the treatment channel may be adapted for accommodating and allowing operation of a non-elongate tool or for a tool that does not require access via the proximal opening 44, and thus this opening may be omitted in such an embodiment.

For example, the tool may comprise a dental laser tool, and such tools are well known in the art. Referring to FIG. 6, for example, the treatment channel 40 may extend to the user end 54 of the handpiece 50, at the proximal opening 44′ thereat, and allow a suitable laser light guide, such as for example one or a plurality of suitable fibers 69, to be passed through the treatment channel 40 to the distal opening 42 and to pass therethrough. (Alternatively it may also be possible to route the fibers 69 via proximal opening 44 when used with the embodiment of FIG. 2.) The proximal end 61 of the optical fiber(s) 69 is optically coupled to a suitable laser radiation generator, for example an Erbium laser light source, via suitable optical couplers. Alternatively, the optical fibers 69 may be provided in the elongate treatment channel 40 of the embodiment of FIG. 2, via the proximal opening 44. Alternatively, the laser tool may comprise a hollow wave guide, coupled to the laser source, and sealed at the distal end 63 by a sapphire tip, which is shaped to direct the laser radiation in the required direction to the area to be treated, for example axially as illustrated in FIG. 6, or at an angle to axis 25, as illustrated in FIG. 6a, where the distal end 63′ is wedge-shaped at a suitable wedge angle. Such a hollow wave guide may be, by way of example, of diameter about 100 micron to about 160 micron.

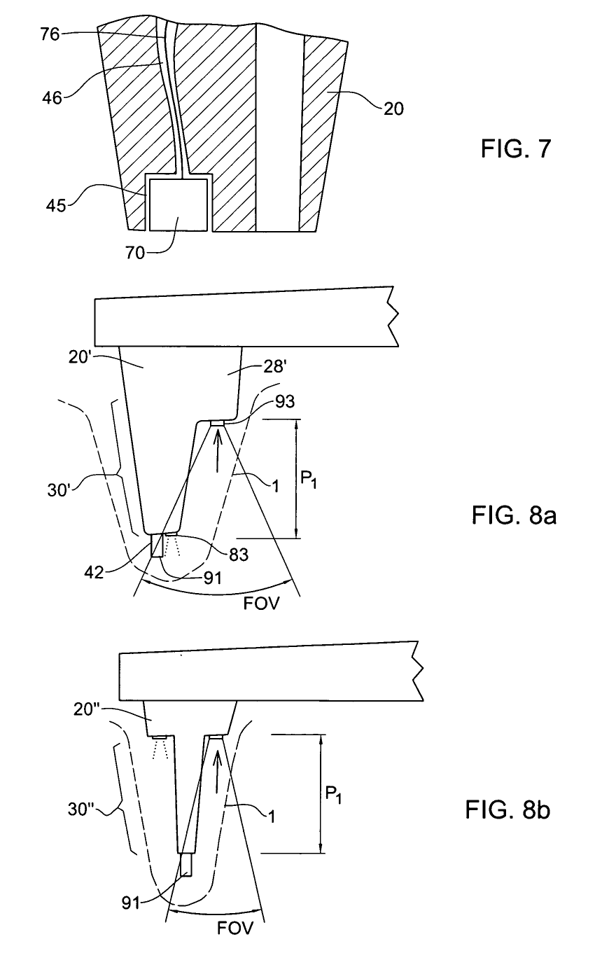

In another example, and referring to FIG. 7, the required tool for root canal treatment may be an ultrasonic tool or an RHF cutting tool, collectively referred to as treatment tool 70, wherein, the treatment channel 40 comprises a recess 45 for accommodating the tool, and a cable channel 46 for passing the cables 76, that provide power and control to the treatment tool 70, between the treatment tool 70 and the user end 54 of the device 100, to be connected from there to a suitable power and control system.

Ultrasonic cutting tools are known in the art and may comprise, for example, a piezo electric or electromagnetic source for providing the high energy vibrations required for operation of the tool. Some examples of such ultrasonic cutting tools may include: the Enac device, produced by Osada (Japan); the Satalec device, produced by Acteon Group, (France); the EMS ultrasonic device, produced by EMS (Switzerland); the Varios 750 device, by NSK (Japan); the Miniendo II device produced by Sybron Dental (USA).

RHF cutting tools are also known, for example diathermic devices (monopolar, bipolar, RHF) and can be used for cutting through dental soft tissues. By way of non limiting example, such a device may include the Erbotom 80 device, produced by ERBE (Germany).

In some embodiments of the invention, several treatment channels may be provided, for accommodating a plurality of tools.

Referring again to FIGS. 2 and 3, the device 100 further comprises an illumination channel 85, comprising a light guide 80, for illuminating the internal desired regions of the intra-oral cavity, such as for example the root canal, during operation of the device 100. Light guide 80 may be in the form of a plurality of optical fibers 81 (only one fiber is schematically illustrated in FIG. 2) accommodated within the shell 29 of the probe member 20, having a proximal end 82 configured for optical coupling to a suitable light source system 200 (FIG. 9), and a distal end 83 in distal portion 30 via which illuminating light from them light source is transmitted to the internal regions of the root canal during operation of said device. Thus, the light guide 80 may comprise a multi-fiber wave guide, having, by way of non-limiting example, a diameter of between about 300 micron to about 350 micron accommodating about 3000 optical fibers or more. The distal end 83 of the fibers may be optically coupled to a suitable lens for focusing the illumination light to any desired part of the internal regions of the root canal. Thus, in this embodiment, the illumination channel 85 comprises the internal volume defined by the shell 29, less the volume taken up by the treatment channel 40 and the image acquisition unit (see below).

Alternatively, the illumination channel may comprise a suitable hollow wave guide optically coupled to one or more LED's, which may optionally be accommodated in the device 100. The LED's may be powered by a suitable power source, e.g. batteries, in the device 100, and/or via an external power source via suitable connectors.

In alternative variations of this embodiment, a plurality of illumination channels may be provided.

Referring again to FIGS. 2 and 3, the device 100 further comprises an image acquisition unit, configured for acquiring images of the internal regions of the root canal illuminated by the illuminating channel 85. In the illustrated embodiment, the image acquisition unit comprises a light collection channel 95, comprising a light guide 90, and the light guide 90 comprises a lumen 99 having a proximal end 92, equipped with suitable lenses, configured for optical coupling to an imaging system 96, such as for example a CCD, and a distal end 93, also equipped with suitable lenses, located in said distal portion 30 for collecting and transmitting light reflected from internal regions of the root canal to the proximal end 92 during operation of said device. In the illustrated embodiment, the imaging system 96 is housed in the hand piece 50 and have its optical axis generally aligned with axis 55, and a suitable optical folding arrangement, such as a mirror or prism 98, may be used to direct light collected from the internal regions of the root canal via distal end 93 to the imaging system 96.

In alternative variations of this embodiment, the treatment channel 40 and the lumens 99 and 38 are not defined by respective real tubes, but rather are defined within the volume enclosed by the cylindrical shell 29, in which different parts of this enclosed volume act as treatment channel 40 and as the lumens 99 and 38. Furthermore, the distal ends 42, 93, 83 of the treatment channel, the light collection channel 95, and the illumination channel 85, respectively, are all comprised at the distal end 33 of the distal portion 30, and thus the width W₁ of the distal end 33 must be sufficiently narrow to allow the distal portion 30 to be inserted into a desired depth with respect to a particular root canal, for example up to about a third, up to about two thirds or up to substantially the full depth of the canal towards the apex. Alternatively, the illumination channel and the light collection channel may be integral one with the other, and comprise a plurality of optical fibers, wherein some of the fibers are used for illumination, while others are used for image acquisition.

The image acquisition unit comprises a field of view (FOV), and the device 100 is configured such that at least during operation of the device, the working end 91 of a particular tool (that is accommodated in the treatment channel) and that projects distally from the distal end of the distal portion 30, is in this field of view. The field of view of the image acquisition unit in this embodiment is about 120°, and this may be achieved, form example, by providing a suitable 120° convex lens at the distal end of the distal portion 30 in optical communication with the image acquisition unit. In alternative variations of this embodiment, the image acquisition unit may have a different field of view, for example greater than about 90°, for example 180°.

Image data collected by the imaging system 96 may be communicated to a suitable image analysis and display unit 300 (FIG. 9), for example a computer, for analysis and display. The imaging system 96 may provide discrete images of the said internal surfaces as required, and/or may provide a sequence of such images in real time providing a video stream that may be viewed by the user of the device and/or any other observer. Optionally, such images may also be recorded in a memory or any suitable recording device. Alternatively, the imaging system 96 may comprise a video camera.

In alternative variations of this embodiment, the imaging system 96 may be accommodated in the probe member 20. For example, the image acquisition system may comprise a CCD or the like, accommodated at or near the distal end of the distal portion 30, and operatively connected to the image acquisition system, for example via cables. In at least some such embodiments, the CCD is disposable together with at least the distal end of the distal portion 30 after use of the device with a patient. Alternatively the imaging system 96 may be accommodated in the handle piece 50, but having its optical axis generally aligned with axis 25, and thus the optical folding arrangement illustrated in FIG. 2 may be omitted from the lumen 99.

By way of non-limiting example, the diameter d of lumen 99 may be from about 300 micron to about 1000 micron.

Alternatively, the light guide 90 may be in the form of a plurality of optical fibers accommodated within the shell 29 of the probe member 20, having a proximal end configured for optical coupling to a suitable imaging system via an optical connector at the user end 54 of the device 100, and having a distal end in distal portion. Thus, the light guide 80 may comprise a multi-fiber wave guide, having, by way of non-limiting example, about 3000 to about 6000 optical fibers. The distal end of the fibers may be optically coupled to a suitable lens for focusing into the fibers the light reflected from the illuminated internal regions of the root canal.

In alternative variations of this embodiment, a plurality of light collection channels may be provided.

Referring to FIGS. 3 and 9 in particular, the device may further optionally comprise at least one auxiliary channel 35 having a proximal end 37 configured for operative connection to at least one of a fluid source and a vacuum source, collectively designated herein at 400, via fluid or vacuum line 39. The auxiliary channel 35, in the for of as lumen 38, further comprises a distal end in said distal portion 30 for providing fluid or a vacuum, respectively, to internal regions of the root canal during operation of said device in root canal treatments, for example. The fluid may be pressurized air or a lubricating, sterilizing or washing liquid, for example, and the vacuum provided by the source 400 allows the treated area to be drained. The lumen 38 may comprise an internal diameter of about 50 micron to about 70 micron, by way of non-limiting example.

By way of non-limiting example, the diameter of auxiliary channel 35 may be between about 55 micron and about 75 micron.

In the embodiment illustrated in FIGS. 2 and 3, the treatment channel 40 and the lumens 99 and 38 are each defined by suitable tubes (not shown) enclosed in a cylindrical shell 29, and the space between the inner surface of the shell 29 and the outer surface of the tubes comprises a plurality of fibers 81 and defines the illuminating channel 85.

By way of non-limiting examples: the diameter W₁ of the probe member 20, in particular of the distal portion 30, may be between about 300 micron and about 1500 micron; axial length D₁ of the distal portion 30 may be between about 15 mm and about 30 mm, or between 18 mm and about 25 mm, or between 10 mm and 15 mm, and the particular range may depend on the depth of the root canal being treated or monitored, and the penetration desired into the root canal for this particular application of the device. The longitudinal dimension D₂ of the probe member 20 may be between about 15 mm and about 30 mm, or between 18 mm and about 25 mm, or between 10 mm and 15 mm, and the particular range may depend on the depth of the root canal being treated or monitored, and the penetration desired into the root canal for this particular application of the device. These dimensions may also be suitable for other applications of the device, for example sinus augmentation procedures, implant procedures and so on, or alternatively, the device may have alternative dimensions more suited for such alternative procedures.

Other arrangements are of course possible.

For example, referring to FIG. 8a, in a modified probe member 20′ the distal ends 42 and 83 of the treatment channel and the illumination channel 85, respectively, are comprised at the distal end 33 of the distal portion 30, while the distal end 93 of the light collection channel 95 is comprised on an enlarged portion 28′ of the probe member 20′. The distal portion 30′ of the modified probe member 20′ comprises the distal end 93, and the enlarged portion 28′ is spaced from the tip or distal end 33 of the probe member 20′ by a dimension P₁, allowing the diameter of the probe member 20′ distal from the enlarged portion to be narrower than at the enlarged portion, which may have the same diameter as that of the distal end 33 of the embodiment of FIG. 2, for example. Thus, the geometry of the distal portion 30 may take advantage of the relatively wider root width at the neck, while maximizing the size of the treatment channel 40 and illumination channel 85. As may be seen, the working end 91 of the tool in the treatment channel 40 is within the field of view FOV of the light collection channel 95. In a further alternative arrangement, the positions of the distal ends 93 and 83 of the light collection channel 95 and the illumination channel 85, respectively, may be swapped, so that the distal end 93 of the light collection channel 95 is now distal with respect to the distal end 83.

In another example illustrated in FIG. 8b, a modified probe member 20″ comprises the distal end 42 of the treatment channel at the distal end 33 of the distal portion 30, while the distal ends 83, 93 of the illumination channel 85 and the light collection channel 95, respectively, are comprised on an enlarged portion 28″ of the probe member 20″. The distal portion 30″ of the modified probe member 20″ comprises the distal ends 83, 93, and the enlarged portion 28″ is spaced from the tip or distal end 33 of the probe member 20″ by a dimension P₁, allowing the diameter of the probe member 20″ distal from the enlarged portion to be narrower than at the enlarged portion, which may have the same diameter as that of the distal end 33 of the embodiment of FIG. 2, for example. Thus, the geometry of the distal portion 30 may take advantage of the relatively wider root width at the neck, while maximizing the size of the treatment channel 40 even more than in the example of FIG. 8a.

Dimension P₁ may be, by way of example, between about 50% and about 70% of the dimension D₁ of the distal portion 30.

The device 100 can therefore form part of a system for implementing procedures associated with the intra-oral cavity, for example treating a root canal, comprising, in addition to said device 100, at least one of: an illumination source 200 operatively connected to the illumination channel 85; an image analysis and display system 300 operatively connected to the light collection channel 95; a fluid and/or vacuum system 400 operatively connected to the auxiliary channel 35; and a suitable tool 150.

The device 100 may be fully disposable, i.e., may be made from materials that render the device disposable after use with one patient or that permit such disposability from an economic perspective, for example. Alternatively the probe member or at least the distal portion thereof are disposable, and are releasably connected to the hand piece 50 or the remainder of the probe member, respectively, in a suitable manner. Alternatively, the device, or the probe member, or at least the distal portion thereof, may be provided with a suitable sheath (not shown) for protecting the device from contamination during use, and the sheath is disposed of after use with a patient.

Alternatively, the device 100 may be sterilizable, for example by autoclaving, and components thereof sensitive to such sterilization are removable therefrom prior to any such operation.

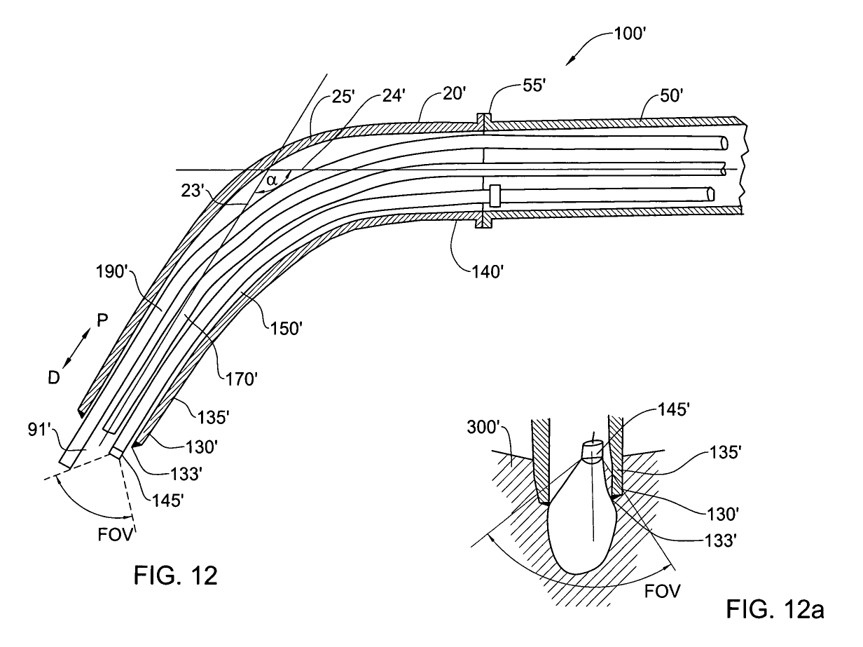

Referring to FIG. 12, a device 100′ according to a second embodiment of the invention comprises all the elements and features of the first embodiment, mutatis mutandis, including its incorporation in system 500 and its uses, with the following differences.

Device 100′, comprises a handpiece 50′ and a probe member 20′ axially mounted thereto and connected to the handpiece 50′ via a luer lock, bayonet fit or any other suitable connection 55′, the probe member 20′ having an elbow 25′ to define angle α between the axis 23′ of the probe at the distal end 130′ and the axis 24′ at the proximal end 140′, which is co-axial with the longitudinal axis of the handpiece 50′. The probe has a central passageway 150′ between the open distal end 130′ and the open proximal end 140′, and this passageway collectively functions as the treatment channel 40 and the lumens 99 and 38 of the embodiment of FIG. 2, mutatis mutandis. In this embodiment, the image acquisition unit 145′ comprises may comprise a CCD or the like, accommodated at or near the distal end of the distal portion 135′ of the probe member 20′, and operatively connected to the respective image acquisition system, for example via cables. In at least some such embodiments, the CCD is disposable together with at least the probe member 20′ after use of the device with a patient.

The image acquisition unit 145′ comprises a field of view (FOV), and the device 100′ is configured such that at least during operation of the device, the working end 91′ of a particular tool 190′ (that is accommodated in the passageway 150′) and that projects distally from the distal end of the distal portion 135′, is in this field of view. The field of view of the image acquisition unit 145′ in this embodiment is about 120°, and this may be achieved, form example, by providing a suitable 120° convex lens at the distal end of the distal portion 135′ in optical communication with the image acquisition unit. In alternative variations of this embodiment, the image acquisition unit may have a different field of view, for example greater than about 90°, for example 180°.

A suitable illumination arrangement 170′ may be provides as in the embodiment of FIG. 2, and additional tools including an irrigation and/or suction catheter may be provided via the passageway 150′.

In this embodiment, the distal end 130′ is also optionally configured for use as a cutting or boring tool. For this purpose, a distal edge 133′ projects beyond the distal end 130′ of the probe, and the distal edge 133′ may be sharp, serrated, or abrasive, and this feature of the device 100′ is used by manually manipulating the device 100′ to provide a material removal action with this edge in contact with a tissue surface, for example. Operation of this tool may be monitored in real time via the image acquisition unit 145′, which keeps the distal edge 133′, i.e., the working end of the tool, in the field of view thereof by retracting the image acquisition unit 145′ into the passageway until the distal edge 133′ comes into view (FIG. 12a). The probe member 20′, or at lest the distal portion 135′, may be made from a transparent material which further aids in monitoring the operation of the edge 133′ with respect to tissue 300′.

In alternative variations of this embodiment, the distal edge defines the distal end of the probe, and the working end of the tool is within the field of view of the image acquisition unit 145′ by retracting the image acquisition unit 145′ into the passageway until the distal edge 133′ comes into view. Alternatively, no retraction may be needed, and the image acquisition unit is provided with a very wide field of view, for example 180°.

The cutting edge feature of the embodiment of FIG. 12 may also be comprised in the embodiment of FIG. 2, mutatis mutandis, and is kept within the field of view of the respective image acquisition unit in a similar manner, mutatis mutandis.

The device 100 and system 500 of the invention may be operated in a number of ways. Operation of the device 100′ and the system incorporating the same is substantially similar, mutatis mutandis.

In monitor mode, the device 100 is used only as an endoscope, for monitoring any desired region of the intraoral cavity or indeed any other part of the anatomy, for example the state of the tooth, of a root canal, or of the gums for example, for providing visual data thereof. This can assist the dental practitioner such as the dentist or surgeon to plan and execute a root canal treatment or other treatment in the oral cavity, or indeed other body parts. In operation, the illumination source is switched on to illuminate the dental or other surfaces that it is desired to inspect, via the illumination channel 85, and the image acquisition unit picks up light reflected from these surfaces and displays the same in the optical display system 300. The configuration of the distal portion 30, in particular its narrow and elongate dimensions are such as to allow the distal end 93 to be within the narrow confines of a root canal or other tight body spaces so that this may be directly imaged by the system 300, and thus the user and/or other parties.

In root canal treatment mode, the device is used as a root canal treatment device with the capability of real time monitoring of the treatment. Here, the device 100 can be coupled with a drill tool for removing the tooth crown. Then, the drill tool is replaced with a suitable file, which is inserted into the treatment channel 40 so that the working end of the file is distally projecting into the root, and the file is used to excise the pulp material and to clean the dentine walls of the root. As more and more pulp is removed, the distal portion 30 of the device 100 is inserted deeper into the root. During all these operations the illumination channel 85 illuminates the interior surfaces of the tooth, and the image acquisition unit picks up light reflected from these surfaces, which are in the filed of view of the image acquisition unit together with the working end of the tool (since the working end of the tool is in contact with or in close proximity to these surfaces during operation thereof) and displays the same in the optical display system 300, as described for the monitor mode, mutatis mutandis, allowing the user to ensure that the root is fully cleaned before progressing to the next step, and without the need for periodic x-rays or other diagnostic procedures, for example.

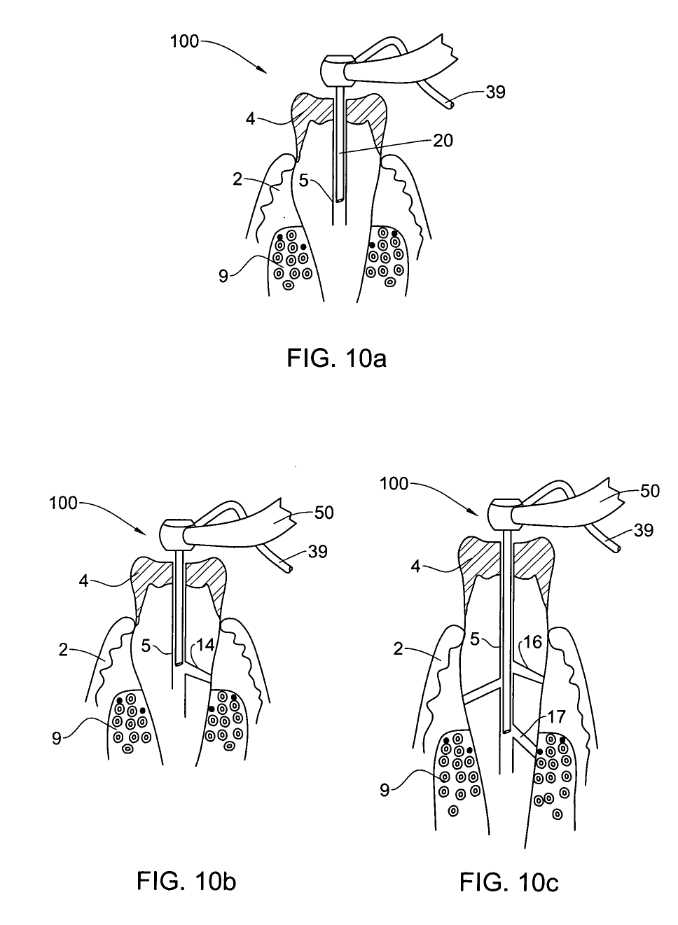

Alternatively, the root may be prepared using an ultrasonic tool, or a RHF tool, or a laser tool, and so on. Referring to FIGS. 10a to 10c, operation of device 100 using a laser tool is illustrated, in which FIG. 10a illustrates a cross sectional view of the dental area being treated, comprising crown 4 and root canal 5, seating in a lower gum bone 9 and margined by gums 2. The probe member 20 of the device 100 is inserted into the root canal 5. The provision of the monitoring or imaging channel in close proximity to the treatment tool, allows for directing the treatment laser radiation to a specific region inside the root canal. For example, as illustrated in FIG. 10b, such monitoring enables identification of a side root canal 14 to thereby allow a dentist to manipulate the device 100, in particular the probe member 20 that carries the laser treatment tool, which may have a wedge shaped distal end) to direct the treatment radiation into canal 14. As shown in the example of FIG. 10c, the laser treatment tool carried in the treatment channel may be appropriately manipulated, as a result of the imaging provide by the device, to direct light to main canal 5 and side canals 16 and 17.

During such operations, the root canal may be rinsed and drained using the auxiliary channel 35 and source 400.

Once the root has been cleaned out to the satisfaction of the dental practitioner, the root may be sealed and filled. For this purpose, the tool that has been used for preparing the root canal is removed, and a suitable sealant, such as for example epoxy resins, zinc oxide eugenol, calcium hydroxide, and so on, may be injected into the root canal via the treatment channel 40. For this purpose, a syringe needle accommodated in the treatment channel may be used for example, the needle being coupled at its proximal end to a syringe containing the sealing agent, and the distal end of the needle projected distally from the distal end of the probe member 20. Then, a suitable filler, such as for example warmed gutta percha or Resilon may be injected into the root canal in a similar manner to that described for the sealant, mutatis mutandis. The sealant and/or filler may be self curing or may require light curing. In the latter case, the illumination system 200 may be configured for providing illumination light of the appropriate wavelength for this curing, for example. During the sealing and filling operations, the user is able to monitor these activities, and adjust the same if necessary.

If necessary, a suitable antiseptic may be provided to the root canal via the treatment channel (for example using another syringe), prior to sealing and filling.

Other root canal procedures are also possible to perform using the device 100. For example, it may be possible to conduct an apicoectomy procedure via the root canal that has been previously prepared, for example as described above, mutatis mutandis. Then, a suitable laser tool or other electrosurgery tool, such as for example an RHF cutting tool, is coupled to the device 100, which is inserted into the root canal until the working distal end of the tool is close to the dental tissues around the apex 7 of the root. The tool can then be operated to cauterize the tissues, and the insertion and alignment of the tool, and operation thereof, may be monitored and control via the monitoring capabilities of the device 100. Similarly, the user can inspect the root tip to ensure that it has been fully treated before sealing and filling the root.

The device and system of the invention may also be used for other dental procedures. For example, the device and system may be used for carrying out procedures relating to periodontal pockets in the gingival tissues, and allow for exploration and/or treatment of the periodontal pockets by means of the monitoring and/or treatment capabilities thereof. The illumination channel and the image acquisition unit may be used for obtaining images of the periodontal pockets, and the treatment channel for providing irrigation thereto, or indeed for providing any other suitable treatment therethrough.

It should be understood that the present invention provides, if desired (based on the imaging) for appropriately manipulating (redirecting) the treatment tool (e.g., laser beam) while inside the root canal. Moreover, the use of the laser-based treatment allows for carrying out the entire root canal treatment (i.e., removing hard tissues inside the root canal) and the canal disinfection procedure.

The device and system of the invention may also be used for other dental, medical or surgical procedures, such as for example a minimally invasive sinus augmentation procedure in which the risks of damaging the sinus membrane are also minimized.

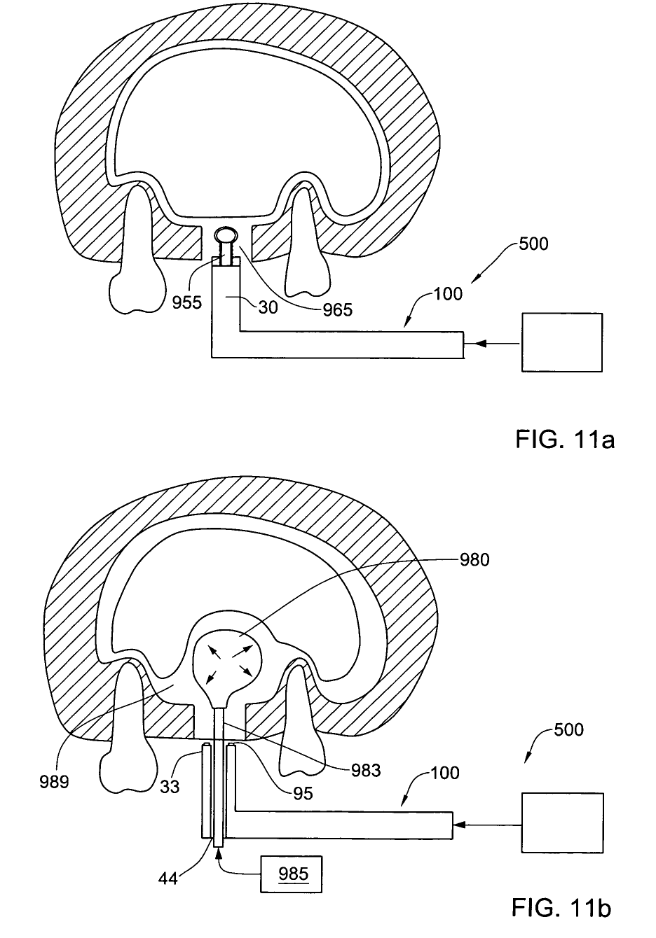

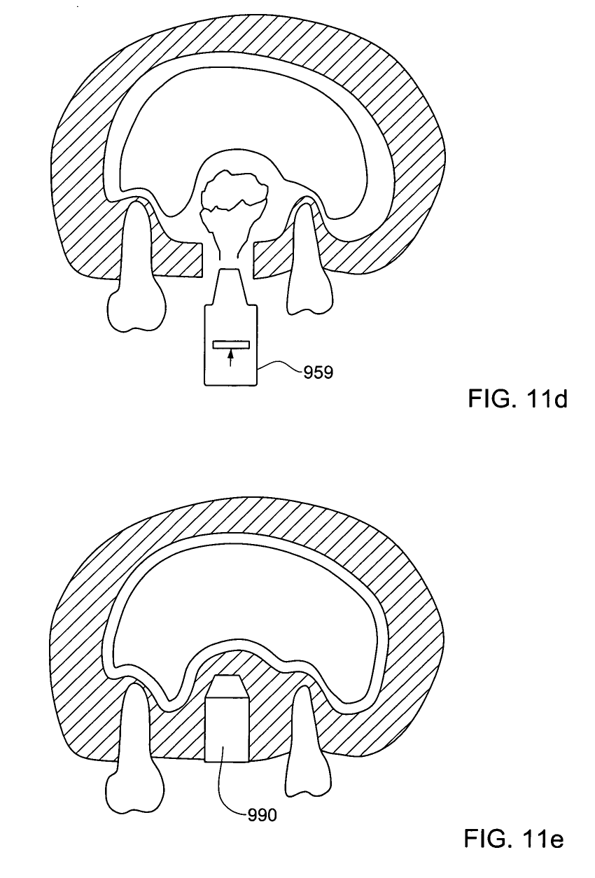

Referring to FIG. 11a, a cross-section of a patients maxilla 900 includes a sinus 910 having a sinus membrane 920. Existing teeth 931, 932 are shown on either side of the implantation site 950 where it is desired to install a dental implant, and where the thickness of the bony wall 960 at the crest of the maxilla is initially insufficient for anchoring the dental implant.

FIGS. 11b to 11e illustrate a sinus augmentation procedure, which also includes a dental implant installation procedure, according to one embodiment of the invention. Referring to FIG. 11b, the first step in the procedure comprises cutting a window 972 in the crest or occlusal-facing gum tissue (not shown) where the implant is to be installed, and the gum tissue may be removed or pulled back. This window 972 is cut using the system 500 and device 100 of the invention according to any one of the embodiments of FIGS. 2 to 10c for example. Alternatively, any suitable traditional tool may be used for creating the window, for example a scalpel.

Then, and referring for example to the embodiment of FIG. 2, the distal portion 30 of the device 100 is brought into proximity with the crest 962 and a suitable tool having a working end 955 such as a dental drill or laser, for example, is provided via treatment channel 40. The tool removes, by drilling, ablation, or any other suitable bone and tissue material removal process, a section of the bony wall 960 to create a channel 965. The material removal operation is under constant monitoring by the surgeon operating the device 100, via the image acquisition system, while the area being cut and monitored is illuminated via the illumination channel 85. Bone tissue is removed until the sinus membrane 920 is exposed, and this point in the procedure is identified in a relatively easy manner since the area being operated on by the working end 955 of tool is in the field of view of the image acquisition unit and thus in constant visual observation by the surgeon via the imaging system, which can provide the surgeon with a magnified video image in real time of this area.

In the next step, illustrated in FIG. 11c, a balloon 980 in the deflated state is inserted into the channel 965, for example via treatment channel 40 (after the material removing tool is removed). For example, the deflated balloon 980 may be accommodated in a cannula 982 that is inserted into the treatment channel 40 from the proximal opening 44. The distal tip 983 of the cannula 982 is distally projected from the distal end 33 of the device 100 so that it comes as close as possible to the membrane 920, being careful not to damage the membrane 920. Such a balloon and cannula arrangement may resemble a Fogarty catheter, but of a size compatible with the size of the treatment channel.

The balloon 980 is then inflated using a suitable saline source 985, with the device 100 held in a particular spatial position with respect to the channel 965, and as saline is provided to the balloon and the balloon inflates, the sinus membrane 920 is deflected in a distal direction towards the interior of the sinus cavity 910. The balloon is preferably made from a transparent material, or at least from a translucent material, and thus, any potential damage to the sinus membrane 920 can be easily spotted by the surgeon during the procedure via the image acquisition unit and imaging system 96, since at least a part of the balloon is in the field of view of the image acquisition unit. In such circumstances the inflation of the balloon may be interrupted, and the balloon removed so that the sinus membrane may be repaired. Otherwise, inflation of the balloon continues, and when the sinus membrane 920 has been sufficiently displaced by the balloon to create a sufficiently large filing space 989, the balloon 980 is deflated carefully, and removed via the treatment channel.

At this point, or earlier, if there is detected a rupture, tear or other damage to the sinus membrane, this may be repaired by overlaying a collagen membrane over the damaged sinus membrane. This repair may also be carried out using the device 100, wherein the collagen membrane is manipulated into place over the damaged area via a suitable tool provided via the treatment channel 40, while monitoring the repair procedure via the image acquisition unit and imaging system 96.

In the next step, a suitable packing material is placed in the space 989, and this may be done in a variety of ways.

In one example, the device 100 is removed from the intra-oral cavity, and a sponge comprising a collagen matrix or other suitable matrix is placed in the space 989. The matrix may be pre-impregnated with BMP (bone morphological protein) or any other suitable bone substitute prior to installing the sponge in the space 989, or alternatively the sponge may be installed in a relatively “dry” configuration, and subsequently the sponge is injected in-situ with BMP or other bone substitute. This injection process may be carried out using the device 100, wherein the syringe needle is brought into proximity with the sponge via the treatment channel 40, at the same time allowing monitoring of the injection process via the image acquisition unit and imaging system 96. Furthermore, the installation of the sponge may also be carried out using the device 100, and for this purpose, a grasping tool such as for example micro tongs or micro-forceps, micro-prongs, scissors, etc, many examples of which are commercially available, may be provided through the treatment channel 40, such that the grasping portion of the tool projects beyond the distal end 33 and holds the sponge, The proximal end of the tool is manipulable from outside the treatment channel to activate and deactivate the tool. Thus, the sponge is maneuvered into the space 989 by manipulating the device 100, the installation procedure being monitored via the image acquisition unit and imaging system 96. In alternative variations of this embodiment, BMP or other liquid bone substitute material is injected directly into the space 989 via the syringe in which the syringe needle is coupled to the treatment channel 40 and projects distally from distal end 33, the injection process being monitored via the image acquisition unit and imaging system 96. In yet other alternative variations of this embodiment, and referring to FIG. 11d, BMP or other liquid bone substitute material is injected directly into the space 989 via a syringe 959 in which the respective syringe needle is inserted into the space 989, without the device 100 (FIG. 11d).

In another example, pieces of allogenic, autogenous, or synthetic bone graft material is inserted into space 989 via the channel 965, and the aforementioned grasping tool may be provided through the treatment channel 40, such that the grasping portion of the tool projects beyond the distal end 33 and holds the graft material. Thus, the graft material is maneuvered into the space 989 by manipulating the device 100, the insertion procedure being monitored via the image acquisition unit and imaging system 96.

In the same manner as the space 989, the channel 965 is also filled with bone graft material, and the window may then be stitched closed, to allow for the sinus augmentation to heal and for the material in the space 989 and channel 965 to become fully integrated with the boney tissues of the maxilla. After the healing process, a suitable hole may be bored into the maxilla at the implantation site in the standard manner, and the implant 990 may be implanted therein in the usual manner (FIG. 11e), followed in due course with a suitable prosthesis mounted to the implant.

Alternatively, rather than filling up the channel 965 completely, the channel 965 may be partially filled with bone graft material, and the implant immediately installed in the channel 965 as part of the sinus augmentation procedure. Then, the bone graft material is allowed to heal, fully integrating with the boney tissues of the maxilla, but at the same time anchoring the implant 990 in place.

The device and system of the invention may also be used for other dental procedures, such as for example for drilling a bore for an implant in the mandibular jaw in which the risks of damaging the inferior alveolar nerve in the mandibular canal are minimized.

In the method claims that follow, alphanumeric characters and Roman numerals used to designate claim steps are provided for convenience only and do not imply any particular order of performing the steps.

Finally, it should be noted that the word “comprising” as used throughout the appended claims is to be interpreted to mean “including but not limited to”.

While there has been shown and disclosed example embodiments in accordance with the invention, it will be appreciated that many changes may be made therein without departing from the spirit of the invention.

1. A method for conducting a procedure in the intra-oral cavity comprising implementing the procedure using at least one suitable tool having a working end, and monitoring the procedure in real time using a suitable imaging system comprising an image acquisition unit, wherein said tool working end and said image acquisition unit are included in a handheld device, and wherein said image acquisition unit and said tool working end are positioned in the hand held device such said tool working end is in the field of view of said image acquisition unit at least during operation of the tool.

2. Method according to claim 1, wherein said procedure comprises a tissue removal operation.

3. Method according to claim 1, wherein the step of implementing said procedure includes using, in turn, each one of a plurality of said tools, each said tool having a respective working end.

4. Method according to claim 3, wherein said procedure is a sinus augmentation procedure applied to a maxilla comprising gum tissue and having a sinus lined with a sinus membrane, the method comprising the steps of:
(a) forming a window in the gum tissue in the maxilla;
(b) providing said device with a first said tool having a first tool working end that is configured for removing tissue;
(c) using said first tool end, removing material from the maxilla to form a channel extending from said window to said sinus membrane, while monitoring said material removal via said image acquisition unit;
(d) providing via said channel with suitable bone graft material in a space between the sinus and the sinus membrane to provide the required sinus augmentation.

5. Method according to claim 4, wherein prior to step (d), said space is formed by displacing said sinus membrane away from said channel.

6. Method according to claim 5, wherein said sinus membrane is displaced away from said channel by inflating a suitable balloon at or near a distal end of said channel.

7. Method according to claim 6, wherein said balloon constitutes said working end of a second said tool, wherein said balloon is one of transparent and translucent, and wherein said balloon inflation procedure is monitored in real time via said image acquisition unit.

8. Method according to claim 7, wherein subsequent to said displacement of said membrane and prior to step (d), said balloon is deflated and removed.

9. Method according to claim 4, wherein step (d) comprises providing a sponge in said space impregnated with said bone graft material.

10. Method according to claim 9, wherein said sponge is manipulated into position in said space via a third said tool having a respective third said working end comprising a grasping function, wherein said sponge is releasably grasped by said third working end until seated in said space.

11. Method according to claim 10, wherein said sponge comprises a collagen matrix, and is pre-impregnated or impregnated in situ in said space.

12. Method according to claim 4, wherein step (d) comprises injecting said bone graft material in said space.

13. Method according to claim 12, wherein a fourth said tool is provided in the form of a syringe having a respective fourth said working end in the form of a needle opening, and wherein said fourth tool is used for injecting said bone graft material into said space via said needle opening, and wherein said injection process is monitored in real time via said image acquisition unit.

14. Method according to claim 4, wherein step (d) further includes filing said channel with suitable bone graft material, and further comprises the step of closing said window and allowing the bone graft material to integrate with the maxilla and heal.

15. Method according to claim 14, further comprising the step of installing a suitable dental implant in said maxilla such that the dental implant is anchored in said sinus augmentation.

16. Method according to claim 4, wherein step (d) further includes partially filing said channel with suitable bone graft material, and concurrently installing a suitable dental implant in said sinus augmentation, and further comprises the step of closing said window around the implant, and allowing the bone graft material to integrate with the maxilla and heal while concurrently anchoring the dental implant in said sinus augmentation.

17. Method according to claim 4, wherein in step (a), said window is formed on a crest portion of the gum.

18. Method according to claim 3, wherein said procedure is a dental implant procedure for a mandibular jaw, and comprises the steps of:
providing said device with a first said tool having a first tool working end that is configured for removing tissue;
using said first tool end, removing material from the mandibular jaw to form a bore configured for receiving the dental implant, while monitoring said material removal via said image acquisition unit.

19. Method according to claim 18, further comprising checking the bore for evidence of possible perforation of the respective mandibular canal and halting said material removal process to minimize or avoid damaging the respective inferior alveolar nerve.

20. Method according to claim 3, wherein said procedure is a root canal procedure, and comprises the steps of:
providing said device with a first said tool having a first tool working end that is configured for removing tissue;
using said first tool end, removing pulp material from the respective root canal, while monitoring said pulp material removal via said image acquisition unit.

21. A device for use in procedures associated with the intra-oral cavity, the device comprising:
a probe member and a handpiece for holding said elongate probe member, said probe member having an elongate distal portion capable of being manipulated in the intra-oral cavity, said probe member comprising:
at least one treatment channel having a distal opening in said elongate distal portion, said at least one treatment channel being configured for enabling operation of a suitable tool via said distal opening, such that in operation of the device, a working portion of said tool projects distally from a distal end of said distal portion;
at least one illumination channel different from said at least one treatment channel and comprising a first light guide having a first proximal end configured for optical coupling to a light source system, and a second distal end in said distal portion for illuminating regions of interest associated with the intra-oral cavity during operation of said device;
image acquisition unit configured for optical coupling to an imaging system, and further configured for collecting and transmitting light reflected from said regions to said proximal end during operation of said device;
wherein said tool working portion is in the field of view of said image acquisition unit at least during operation of the device.

22. A device according to claim 21, wherein said handpiece comprises a longitudinal axis set at an angle α with respect to a longitudinal axis of said probing member, wherein said angle α is between about 45° and about 135°.

23. A device according to claim 21, wherein said field of view is greater than about 90 degrees.

24. A device according to claim 21, wherein said field of view is about 120 degrees.

25. A device according to claim 21, wherein said imaging acquisition unit comprises a CCD or the like located in said probe member at or near said distal end.

26. A device according to claim 21 comprising a said tool accommodated in said treatment channel wherein said working portion of said tool projects distally from a distal end of said distal portion.

27. A device according to claim 21, wherein said distal end of said probe member is configured as a tool, and wherein during operation of said distal end as a tool, said image acquisition unit is positioned in the device such that said distal end is in the field of view of said image acquisition unit.

28. A system for use in procedures associated with the intra-oral cavity, comprising:
(A) a device comprising a probe member and a handpiece for holding said elongate probe member, said probe member having an elongate distal portion capable of being manipulated in the intra-oral cavity, said probe member comprising:
at least one treatment channel having a distal opening in said elongate distal portion, said at least one treatment channel being configured for enabling operation of a suitable tool via said distal opening, such that in operation of the device, a working portion of said tool projects distally from a distal end of said distal portion;
at least one illumination channel different from said at least one treatment channel and comprising a first light guide having a first proximal end configured for optical coupling to a light source system, and a second distal end in said distal portion for illuminating regions of interest associated with the intra-oral cavity during operation of said device;
image acquisition unit configured for collecting and transmitting light reflected from said regions to said proximal end during operation of said device;
wherein said tool working portion is in the field of view of said image acquisition unit at least during operation of the device.
(B) a light source system coupled to said proximal end of said first illumination system;
(C) an imaging system comprising a display unit coupled to said image acquisition unit;
(D) a said tool accommodated in said treatment channel wherein said working portion of said tool projects distally from a distal end of said distal portion.
